(19) United States
(12) Patent Application Publication
(10) Pub. No.: US 2026/0367436 A1
(43) Pub. Date: Nov. 19, 2026

(54) MEDICAL DISPLAY APPARATUS

(71) Applicants: Beijing BOE Display Technology Co., Ltd., Beijing (CN); BOE Technology Group Co., Ltd., Beijing (CN)

(72) Inventors: Xin GAI, Beijing (CN); Wei YUAN, Beijing (CN); Hongsheng GU, Beijing (CN); Lijie BAO, Beijing (CN); Hetao WANG, Beijing (CN); Chunlei XIAO, Beijing (CN); Wenqian LUO, Beijing (CN); Jingrui REN, Beijing (CN); Qian CHENG, Beijing (CN)

(21) Appl. No.: 19/476,124

(22) PCT Filed: Jun. 12, 2024
(86) PCT No.: PCT/CN2024/098741

§ 371 Date: Oct. 16, 2025

(30) Foreign Application Priority Data
Jul. 31, 2023 (CN) 202310956733.1

(51) Int. Cl. G02F 1/1333; A61B 5/00; G02F 1/1335; G02F 1/13357; G09G 3/34

(52) U.S. Cl. CPC G02F 1/133317; A61B 5/7445; G02F 1/133314; G02F 1/133512; G02F 1/133608; G02F 1/133615; G09G 3/342; G02F 2201/58; G09G 2320/0686; G09G 2320/0693; G09G 2360/145; G09G 2380/08

(57) ABSTRACT

A medical display includes: a display module (100) including a normal light emitting region (110) and an auxiliary light emitting region (120) on a side of the normal light emitting region (110); a frame (200) configured to fix the display module (100) and including a fixing region (240), wherein an orthographic projection of the fixing region (240) in a thickness direction at least partially covers the auxiliary light emitting region (120); and a sensor in the fixing region (240) of the frame (200) and directly facing the auxiliary light emitting region (120), and configured to receive a light signal emitted from the auxiliary light emitting region (120).

### TECHNICAL FIELD

The present disclosure relates to the field of surgical robot technology, in particular to a medical display apparatus.

### BACKGROUND

In recent years, due to the impact of domestic and international epidemics, medical instruments have developed rapidly, liquid crystal display panels are used important interactive interfaces of the medical instruments, and terminal applications have increasingly high requirements for the stability of display screens. A medical display product may be classified into three major types, that is, a monitoring type product, a diagnostic type product and a consultation operation type product, wherein the diagnostic type display product has the strictest requirement. The diagnostic type display has high standard for the delivery quality and strict requirement on the detection standard for the image quality during long-term use, for example, the brightness and curve of the diagnostic type display are periodically corrected. However, when correcting the existing medical display, a sensor pops up from a frame to be aligned with an original display region, and after a test pattern displayed in the original display region is received by the sensor, the sensor retracts into the frame, which may cause the following problems: it is necessary to provide a mechanical transmission structure to achieve the movement of the sensor, the mechanical transmission structure is easily damaged and the provision of the mechanical transmission structure increases a thickness of the frame.

### SUMMARY

An object of the present disclosure is to provide a medical display that avoids the use of a mechanical transmission structure while correcting for brightness and curve.

According to the embodiments of the present disclosure, a medical display apparatus is provided, including: a display module including a normal light emitting region and an auxiliary light emitting region on a side of the normal light emitting region; a frame configured to fix the display module and including a fixing region, wherein an orthographic projection of the fixing region along a thickness direction at least partially covers the auxiliary light emitting region; and a sensor in the fixing region of the frame and directly facing the auxiliary light emitting region, and configured to receive a light signal emitted from the auxiliary light emitting region.

In some implementations, the display module includes a backlight source module extending from the normal light emitting region to the auxiliary light emitting region, and a portion of the backlight source module in the light emitting region and another portion of the backlight source module in the auxiliary light emitting region are configured to be turned on or off synchronously.

In some implementations, the display module further includes a display panel on the backlight source module and extending from the normal light emitting region to the auxiliary light emitting region, and the sensor faces a portion of the display panel in the auxiliary light emitting region to receive a light signal emitted from a whole structure formed by the backlight source module and the display panel.

In some implementations, the display panel includes a first display unit and a second display unit, the first display unit is in the normal light emitting region, the second display unit is in the auxiliary light emitting region, and the first display unit and the second display unit operate synchronously or independently.

In some implementations, the medical display apparatus has a normal display state and a detection state; in a case where the medical display apparatus is in the normal display state, the first display unit is in a light-transmitting state, light emitted from the backlight source module is emitted outwards by transmitting through the first display unit, and the second display unit is in a light-shielding state, and light emitted from the backlight source module is not transmitted through the second display unit; and in a case where the medical display is in the detection state, the second display unit is in a light-transmitting state, and light emitted from the backlight source module is emitted outwards by transmitting through the second display unit and received by the sensor.

In some implementations, the frame includes a light-shielding plate on a side of the display panel away from the backlight source module, and a minimum distance from an edge of the light-shielding plate to the first display unit is greater than or equal to 0.1 mm and/or less than or equal to 5 mm.

In some implementations, the display module further includes a display panel on the backlight source module and in the normal light emitting region, an orthographic projection of the display panel on the backlight source module does not overlap with an orthographic projection of the auxiliary light emitting region on the backlight source module, and the sensor faces a portion of the backlight plate in the auxiliary light emitting region to receive a light signal emitted from the backlight source module.

In some implementations, the frame includes a housing, the housing includes a fixing plate located outside the display module, an orthographic projection of the fixing plate in the thickness direction is at least partially located in the auxiliary light emitting region, a portion of the fixing plate in the auxiliary light emitting region serves as the fixing region, and the sensor is on the fixing plate.

In some implementations, the frame includes a housing and an auxiliary housing, the auxiliary housing includes an extension unit and a fixing unit connected to each other, the extension unit is in the housing, and the fixing unit is exposed outside the housing; and an orthographic projection of the fixing unit in the thickness direction is at least partially located in the auxiliary light emitting region, the fixing unit serves as the fixing region, and the sensor is on the fixing unit.

In some implementations, the sensor is on the fixing plate, and the fixing plate and the display module are parallel to each other; or the sensor is on the fixing unit, and the fixing unit and the display module are parallel to each other.

In some implementations, the display module includes a plurality of pixels, each pixel including sub-pixel units for displaying different colors; and a detection range of the sensor in a light emitting layer covers all sub-pixel units in at least one pixel.

In some implementations, an angle between an edge of the sensor and an edge of the auxiliary light emitting region is greater than or equal to 100° and less than or equal to 180°; and/or a minimum distance from the sensor to the display module is greater than or equal to 0.2 mm and less than or equal to 5 mm; and/or a minimum distance from an edge of an orthographic projection of the sensor on the display module to the edge of the auxiliary light emitting region is greater than or equal to 0.2 mm and less than or equal to 8 mm; and/or a length from an end of the auxiliary light emitting region away from the normal light emitting region to an edge of the normal light emitting region is greater than or equal to 1 mm and less than or equal to 15 mm.

The technical solutions provided by the embodiments of the present disclosure have the following beneficial technical effects:

In the present disclosure, the auxiliary light emitting region is provided, the sensor is fixed on the frame and faces the auxiliary light emitting region. When the apparatus is normally used, the auxiliary light emitting region displays a black picture, and the normal light emitting region is in normal use; during the test, the auxiliary light emitting region displays a test pattern, and the sensor receives and calibrates the test pattern. Therefore, it is unnecessary to provide the mechanical transmission structure, and therefore, the problems of damage of the mechanical transmission structure and the great thickness of the frame can be avoided. Meanwhile, the problem that the original display region is required to be changed into displaying the test pattern during the detection is avoided, so that the conventional normal light emitting region can be always in normal display without changing the displayed picture.

### BRIEF DESCRIPTION OF DRAWINGS

In order to more clearly illustrate the technical solutions in the embodiments of the present disclosure, the drawings used for describing the embodiments will be briefly described below. Obviously, the drawings in the following description are only some embodiments of the present disclosure, and other drawings may be obtained by one of ordinary skill in the art according to the drawings without creative efforts.

FIG. 1 is a schematic diagram of a structure of a medical display according to an embodiment of the present disclosure.

FIG. 2 is a cross-sectional view of a sensor of a conventional medical display in a non-operation state.

FIG. 3 is a cross-sectional view of a sensor of a conventional medical display in an operation state.

FIG. 4 is a schematic diagram of a structure of a display module according to an embodiment of the present disclosure.

FIG. 5 is a cross-sectional view of a medical display according to an embodiment of the present disclosure.

FIG. 6 is a cross-sectional view of a medical display according to an embodiment of the present disclosure.

FIG. 7 is a cross-sectional view of a medical display according to an embodiment of the present disclosure.

FIG. 8 is a schematic structural diagram illustrating a distance between a sensor and a display module according to an embodiment of the present disclosure.

Description of the reference numerals:

| | | | |
|---|---|---|---|
| 100 | Display module | 110 | Normal light emitting region |
| 120 | Auxiliary light emitting | 200 | Frame |
| | region | | |
| 300 | Sensor | 130 | Backlight source module |
| 140 | Display panel | 141 | First display unit |
| 142 | Second display unit | 210 | Light-shielding plate |
| 220 | Housing | 221 | Fixing plate |
| 230 | Auxiliary housing | 231 | Extension unit |
| 232 | Fixing unit | 240 | Fixing region |
| 131 | Back plate | 132 | Light guide plate |
| 133 | Heat radiation plate | | |

### DETAIL DESCRIPTION OF EMBODIMENTS

The exemplary embodiments will be described in detail here, and examples of the embodiments are illustrated in the accompanying drawings. The following description refers to the accompanying drawings in which the same reference numbers in different drawings represent the same or similar elements unless otherwise indicated. The implementations described in the exemplary embodiments below do not represent all implementations consistent with the present disclosure. Rather, they are merely examples of apparatus and methods consistent with certain aspects of the present disclosure, as detailed in the appended claims.

The terminology used herein is for the purpose of describing particular embodiments only and is not intended to be limiting of the present disclosure. Unless defined otherwise, technical or scientific terms used herein shall have the ordinary meaning as understood by one of ordinary skill in the art to which the present disclosure belongs. The use of the terms “a” or “an” and similar referents in the description and the claims of the present disclosure do not denote a limitation of quantity, but rather denote the presence of at least one. “A plurality” denotes two or more. The term of “comprising/comprise” or “including/include”, or the like, means that the element or item preceding the “comprising/comprise” or “including/include” contains the element or item listed after the “comprising/comprise” or “including/include” and its equivalent, but does not exclude other elements or items. The term “connected”, “coupled”, or the like is not limited to physical or mechanical connections, but may include electrical connections, whether direct or indirect connections. The terms “upper” and/or “lower”, and the like are used for convenience of description and are not limited to a certain position or spatial orientation. As used in this specification and the appended claims of the present disclosure, the singular forms “a”, “an”, and “the” are intended to include the plural forms as well, unless the context clearly indicates otherwise. It should also be understood that the term “and/or” as used herein refers to and encompasses any one and all possible combinations of one or more of the associated listed items.

A medical display is an important apparatus in the field of medical imaging, is mainly used for displaying medical pictures, and can take the place of films and achieve the effect of “soft-copy reading” for doctors. However, in the use of the medical display, the performance of a liquid crystal backlight deteriorates with time, so that the brightness of the display is reduced and the gray scale of the display does not conform to the DICOM (digital imaging and communications in medicine) standard. The DICOM standard is one of the technical standards that the medical display must meet, that is, the DICOM standard is the digital imaging and communications in medicine. The DICOM standard defines a medical image format that may be used for data exchange with a quality meeting the clinical needs. The DICOM standard is widely used in the diagnostic equipment of the radiotherapeutics, the cardiovascular imaging and the radiological diagnosis and radiotherapy and in other medical fields such as the ophthalmology and the dentistry.

When the brightness of the display is reduced and the gray scale of the display does not conform to the DICOM standard, the medical display usually needs to measure the brightness by using a brightness meter and corresponding software and calibrate the brightness. The common calibration method includes a manual calibration by using an external brightness meter and an automatic calibration by using an internal brightness meter.

Typically, the DICOM has been calibrated already when the medical display leaves factory. The calibration process includes: a brightness gray scale curve of the display is measured by a professional brightness measuring instrument, then the operation and data processing is performed through a computer according to the highest brightness and the lowest brightness of a screen and a DICOM curve calculation method, and then the calibrated standard DICOM curve is written into a memory of the medical display for use.

In order to ensure the long-term stability of the medical display, it is required that the medical display must be detected and calibrated for the brightness and the DICOM curve after a period of use.

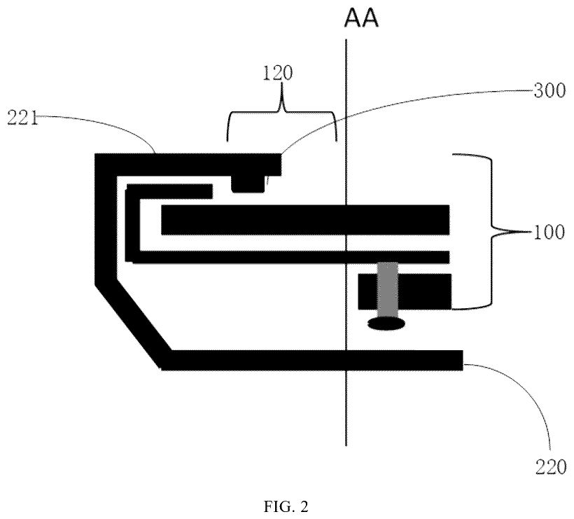

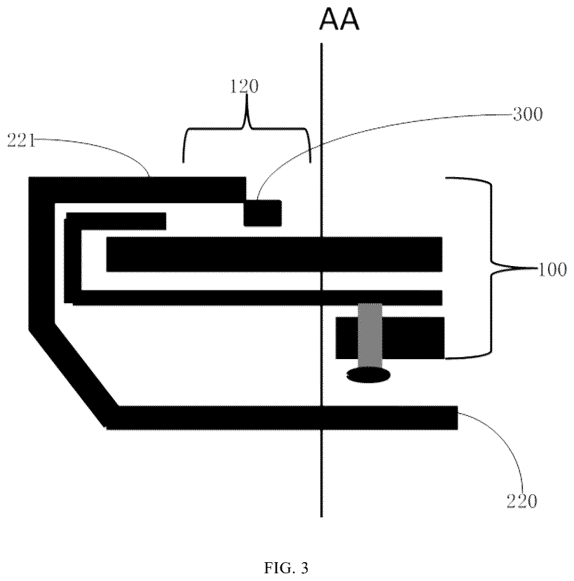

As shown in FIGS. 2 and 3, the medical display generally includes a display module 100, a frame 200, and a sensor 300 and the like. The display module is configured to display medical pictures. The sensor 300 is configured to receive a light signal. The frame 200 is configured to fix the display module 100 and the sensor 300. The sensor 300 is slidably disposed on the frame 200 and may move relative to the display module 100. When the medical display is in normal display, the sensor is located inside the frame 200. When the medical display is corrected for the DICOM, a test pattern is displayed in at least a partial region of the display module 100. At least a portion of the sensor 300 (including at least a photosensitive portion for receiving a light signal) extents from the frame 200 and is aligned with a region of the display module 100 for displaying the test pattern, and then retracts inside the frame 200. At this time, the sensor 300 receives the test pattern displayed by the display module 100. A controller compares the information received by the sensor 300 with the standard DICOM curve written into the memory of the medical display, and then adjusts the display module 100 according to the comparison result. It should be noted that the test pattern is clearly different from the medical image.

This causes following several problems: firstly, a mechanical transmission structure is required to drive the sensor 300 to move, where on one hand, the mechanical transmission structure is easily damaged and on the other hand, the mechanical transmission structure has certain thickness and therefore, occupies a certain space inside the frame, which increases the thickness of the frame 200; secondly, during the detection, it is necessary to display the test pattern on the display module 100, so that during the measurement, at least a part of the display module 100 needs to display the test pattern, in other words, the display module 100 cannot display the complete medical image.

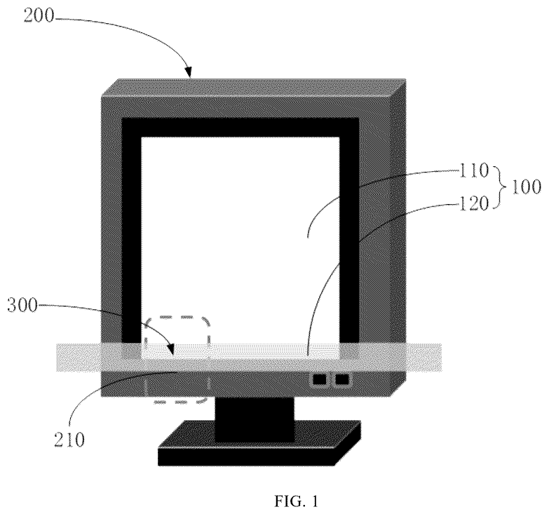

As shown in FIG. 1, the present disclosure provides a medical display including: a display module 100, a frame 200 and a sensor 300, where the display module 100 includes a normal light emitting region 110 and an auxiliary light emitting region 120, and the auxiliary light emitting region 120 is disposed on a side of the normal light emitting region 110; the frame 200 is configured to fix the display module 100, the frame 200 includes a fixing region 240, and an orthographic projection of the fixing region 240 along a thickness direction (a direction perpendicular to a display panel of the display module 100) at least partially covers the auxiliary light emitting region 120; and the sensor 300 is disposed in the fixing region 240 and directly faces (is directly opposite to) the auxiliary light emitting region 120, and the sensor 300 is configured to receive a light signal emitted from the auxiliary light emitting region 120.

In the present disclosure, the auxiliary light emitting region 120 is provided and the sensor 300 is fixed on the frame 200 and directly faces the auxiliary light emitting region 120. During the normal display, the auxiliary light emitting region 120 does not display the picture, and the normal light emitting region 110 normally operates. When the correction of DICOM is required, the auxiliary light emitting region 120 displays the test pattern, and the sensor 300 receives the test pattern and calibrates the normal light emitting region 110.

Therefore, it is unnecessary to provide the mechanical transmission structure, and therefore, the problems of damage of the mechanical transmission structure and the great thickness of the frame 200 can be avoided. Meanwhile, the problem that the original display region is required to be changed into displaying the test pattern during the detection is avoided, so that the existing normal light emitting region 110 can normally operates. The case that the sensor 300 moves under the driving of the mechanical transmission structure is avoided.

In addition, the sensor 300 is provided directly towards (directly faces) the auxiliary light emitting region 120, which can ensure that the sensor 300 can directly obtain the brightness information of a backlight source module 130. It can be understood that the sensor 300 faces the backlight source module 130 to directly obtain the information of the display module 100 without any information loss, so that more fine adjustment can be performed.

With such the arrangement, the problem of low detection precision caused by the fact that the sensor 300 is arranged on a side of the display module 100 and the information of the display module 100 is reflected to the sensor 300 by a light guide unit is avoided.

In this embodiment, based on the above description, the display module 100 includes the backlight source module 130 extending from the normal light emitting region 110 to the auxiliary light emitting region 120, and a portion of the backlight source module 130 in the light emitting region and another portion of the backlight source module 130 in the auxiliary light emitting region 120 are turned on or off synchronously. The backlight source module 130 includes a back plate 131 for supporting the components thereon and homogenizing light, a light guide plate 132 for homogenizing light, a light emitting source for emitting light, and a heat dissipating plate 133, and the like.

In the present disclosure, preferably, the backlight source module 130 has a whole structure. With such arrangement of the backlight source module 130, it can be ensured that a portion of the backlight source module 130 in the normal light emitting region and another portion of the backlight source module 130 in the auxiliary light emitting region 120 are turned on or off synchronously, so that the brightness of the auxiliary light emitting region 120 can reflect the brightness of the normal light emitting region 110 more accurately, and the production cost of the whole backlight source module 130 is lower, which has a cost advantage. Alternatively, in other embodiments, the backlight source module 130 may be formed by combining two backlight source units provided in the normal light emitting region 110 and the auxiliary light emitting region 120. Any embodiment in which the portions of the backlight source module 130 in the normal light emitting region 110 and the auxiliary light emitting region 120 are turned on or off synchronously is within the protection scope of the present disclosure.

In this embodiment, the sensor 300 is disposed facing the auxiliary light emitting region 120 for receiving the brightness information of the backlight source module 130 in the auxiliary light emitting region 120, and the brightness of the normal light emitting region 110 can be estimated according to the brightness information of the backlight source module 130 in the auxiliary light emitting region 120. The reason is that since the portions of the backlight source module 130 in the normal light emitting region and the auxiliary light emitting region 120 are turned on or off synchronously, and thus the signal detected in the auxiliary light emitting region 120 can well reflect the brightness information of the normal light emitting region 110. In summary, the brightness of the normal light emitting region 110 can be adjusted according to the detected brightness information of the auxiliary light emitting region 120.

In a case where the backlight source module 130 has a whole structure, the sensor 300 directly reads the brightness information of the portion of the backlight source module 130 located in the auxiliary light emitting region 120, and directly adjusts the brightness of the whole backlight source module 130 according to the brightness information. In a case where the backlight source module 130 is formed by combining two backlight source units, the sensor 300 reads the brightness information of the backlight source unit in the auxiliary light emitting region 120, and then synchronously adjusts the brightness of the two backlight source units according to the brightness information.

In this embodiment, based on the description of the backlight source module 130, the display module 100 further includes a display panel 140 disposed on the backlight source module 130 and extending from the normal light emitting region 110 to the auxiliary light emitting region 120, and the sensor 300 faces the display panel 140 in the auxiliary light emitting region 120 to receive a light signal emitted from a whole structure formed by the backlight source module 130 and the display panel 140.

The normal DICOM measurement is to measure the light signal emitted from the whole structure formed by the backlight source module 130 and the display panel 140. After the sensor 300 receives the light signal emitted from the whole structure formed by the backlight source module 130 and the display panel 140, the DICOM curve of the normal light emitting region 110 can be detected and corrected through comparison.

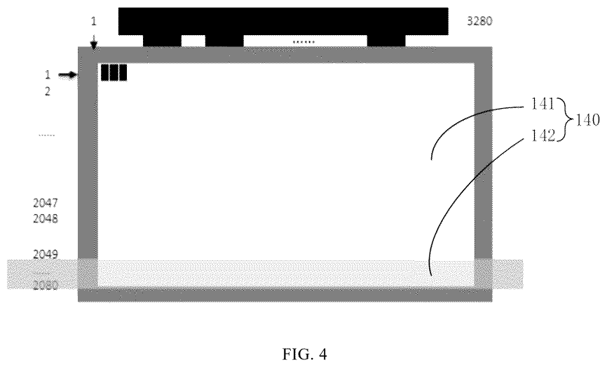

In the present embodiment, referring to FIG. 4, the display panel 140 includes a first display unit 141 and a second display unit 142, the first display unit 141 is disposed in the normal light emitting region 110, the second display unit 142 is disposed in the auxiliary light emitting region 120, and the first display unit 141 and the second display unit 142 operate synchronously or independently.

The second display unit 142 may display in synchronization with the first display unit 141 when the first display unit 141 displays normally. Alternatively, the second display unit 142 may display in a black screen when the first display unit 141 displays normally. When the first display unit 141 and the second display unit 142 operate and display synchronously, the second display unit 142 may expand the visual range of the first display unit 141 to a certain extent, so as to obtain a better display effect. When the second display unit 142 displays in the black screen, the second display unit 142 and the frame 200 are visually integrated together, thereby obtaining a good visual effect. In summary, the respective display of the first display unit 141 and the second display unit 142 may be suitable for various situations.

Based on the above embodiment, the medical display has a normal display state and a detection state. When the medical display is in the normal display state, the first display unit 141 is in a light-transmitting state, and light emitted from the backlight source module 130 may be emitted outwards by transmitting through the first display unit 141; the second display unit 142 is in a light-shielding state, and light emitted from the backlight source module 130 cannot be transmitted through the second display unit 142. When the medical display is in the detection state, the second display unit 142 is in a light-transmitting state, and light emitted from the backlight source module 130 may be emitted outwards by transmitting through the second display unit 142 and received by the sensor 300.

In the normal display state, light emitted from the backlight source module 130 may be emitted outward by transmitting through the first display unit 141, and cannot transmit through the second display unit 142, so that the first display unit 141 normally displays, thereby realizing a predetermined function of the product. When the second display unit 142 displays in the black screen, the second display unit 142 and the frame 200 are integrated together, thereby obtaining a good visual effect.

In the detection state, the second display unit 142 is in a light-transmitting state, and light emitted from the backlight source module 130 may be emitted outwards by transmitting through the second display unit 142 and received by the sensor 300, and at this time, the normal operation of the first display unit 141 is not affected by the operation of the second display unit 142.

In summary, the normal operation of the first display unit 141 is not affected in both the normal display state and the detection state, so that the normal use of the product is not disturbed. Compared with the traditional detection mode, the apparatus in the present disclosure may provide better use experience.

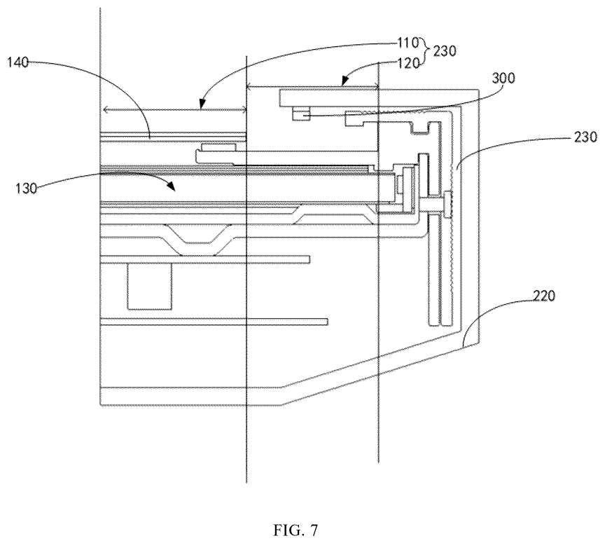

In another embodiment, as shown in FIG. 7, the display panel 140 is located in the normal light emitting region 110, and an orthographic projection of the display panel 140 on the backlight source module 130 does not overlap with an orthographic projection of the auxiliary light emitting region 120 on the backlight source module 130. The sensor 300 faces the backlight plate (a portion of the backlight source module 130) located in the auxiliary light emitting region 120 to receive the light signal emitted from the backlight source module 130.

Since the orthographic projection of the display panel 140 on the backlight source module 130 does not overlap with the orthographic projection of the auxiliary light emitting region 120 on the backlight source module 130, the display panel 140 is not located in the auxiliary light emitting region 120, and the sensor 300 receives the light signal emitted from the backlight source module 130. With such arrangement, the medical display may adjust the portion of the backlight source module 130 in the normal light emitting region 110 by detecting a portion of the backlight source module 130 in the auxiliary light emitting region 120, so as to obtain the better display effect.

It should be noted that when only the portion of backlight source module 130 is disposed in the auxiliary light emitting region 120, the brightness of the backlight source module 130 is measured for adjustment. In a case where the backlight source module 130 and the display panel 140 are both disposed in the auxiliary light emitting region 120, the brightness and the gray scale of the auxiliary light emitting region 120 are measured.

Both the above measurement modes are embodiments of the present disclosure and belong to the protection scope of the present disclosure.

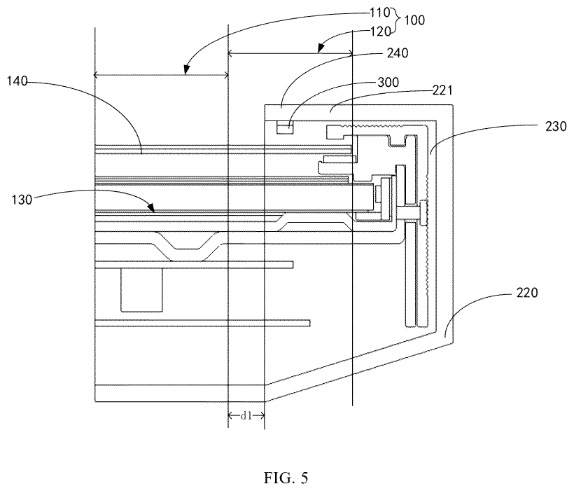

In this embodiment, as shown in FIG. 5, the frame 200 includes a light-shielding plate 210 located on a side of the display panel 140 away from the backlight source module 130, and a minimum distance d1 from an edge of the light-shielding plate 210 to the first display unit 141 is greater than or equal to 0.1 mm and less than or equal to 5 mm. Alternatively, in other embodiments, the minimum distance d1 may be only greater than or equal to 0.1 mm, or only less than or equal to 5 mm.

It is found in practical tests that, by providing a certain distance between the edge of the light-shielding plate 210 and the first display unit 141 an installation margin may be reserved for installation, facilitating the subsequent installation. If no margin is reserved or the margin is too less, there may be a situation where the light-shielding plate 210 shields the first display unit 141. If the reserved margin is too large, it will cause waste and the edge of the light-shielding plate 210 is too far away from the first display unit 141, resulting in bad appearance.

It is found through a lot of experiments that when the minimum distance d1 from the edge of the light-shielding plate 210 to the first display unit 141 is less than or equal to 0.1 mm, it is difficult for the installation and the first display unit 141 is easily shielded. When the minimum distance d1 from the edge of the light-shielding plate 210 to the first display unit 141 is greater than or equal to 5 mm, the waste and the bad appearance may be caused.

Preferably, the minimum distance from the edge of the light-shielding plate 210 to the first display unit 141 is greater than or equal to 0.5 mm and less than or equal to 2 mm. The minimum distance from the edge of the light-shielding plate 210 to the first display unit 141 is in the range of greater than or equal to 0.5 mm and less than or equal to 2 mm, as a good range for the installation, which does not cause the installation difficulty or the first display unit 141 to be shielded, and does not cause the waste but causes the good appearance.

In this embodiment, the minimum distance d1 is 1 mm. In other embodiments, the minimum distance d1 may alternatively be 1.2 mm, or 1.5 mm, or the like.

As shown in FIG. 5, the frame includes a housing 220, the housing 220 includes a fixing plate 221 located outside the display module 100, an orthographic projection of the fixing plate 221 in a thickness direction thereof (that is, on the display panel of the display module 100) is at least partially located in the auxiliary light emitting region 120, and a portion of the fixing plate 221 in the auxiliary light emitting region 120 serves as the fixing region 240, and the sensor 300 is disposed on the fixing plate 221.

It should be noted that the fixing region 240 is at least a part of the fixing plate 221, and the light-shielding plate 210 serves as the fixing plate 221, or is at least a part of the fixing plate 221.

At least part of the orthographic projection of the fixing plate 221 in the thickness direction thereof is located in the auxiliary light emitting region 120, and the sensor 300 fixed on the fixing plate 221 directly faces the portion of the display module 100 in the auxiliary light emitting region 120, so that the sensor 300 is disposed between the auxiliary light emitting region 120 and the fixing plate 221 and directly faces the display module 100, that is, the sensor 300 is completely shielded by the fixing plate 221. Thus, the sensor 300 is not visible from the outside, a hidden design is realized, and the detection function is realized while the aesthetics of the apparatus is maintained.

In this embodiment, the fixing plate 221 is disposed parallel to the display module 100. The fixing plate 221 is disposed parallel to the display module 100, so that the sensor 300 fixed on the fixing plate 221 directly faces the display module 100 to collect the light signal from the display module 100. The sensor 300 directly faces the display module 100, so that the light signal can be well received from the display module 100. Meanwhile, the fixing plate 221 being disposed parallel to the display module 100 can ensure the appearance of the display module 100 to be exquisite and consistent.

It should be noted that in this embodiment, an auxiliary housing 230 is further provided in the housing 220, and the auxiliary housing 230 cooperates with the housing 220 to fix the display module 100.

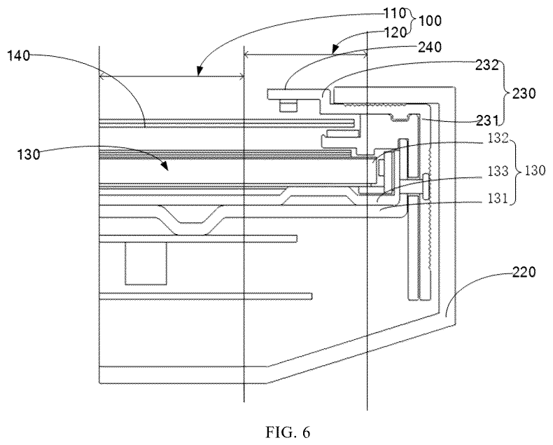

In another embodiment, as shown in FIG. 6, the frame 200 includes a housing 220 and an auxiliary housing 230, the auxiliary housing 230 includes an extension unit 231 and a fixing unit 232 connected to each other, the extension unit 231 is disposed in the housing 220, and the fixing unit 232 is exposed outside the housing 220. An orthographic projection of the fixing unit 232 in the thickness direction thereof (that is, on the display panel of the display module) is at least partially located in the auxiliary light emitting region 120, the fixing unit 232 serves as the fixing region 240, and the sensor 300 is disposed on the fixing unit 232.

It should be noted that in the present embodiment, the fixing region 240 is at least a part of the fixing unit 232, the light-shielding plate 210 may be understood as the fixing unit 232, or the light-shielding plate 210 may be understood as a combination of the fixing unit 232 and at least a part of the frame 200.

The extension unit 231 is disposed in the housing 220 and connected to the fixing unit 232, and supports the fixing unit 232, so that the sensor 300 is not disposed on the housing 220 but only disposed on the fixing unit 232 of the auxiliary housing 230.

Further, the fixing unit 232 is disposed parallel to the display module 100. The fixing unit 232 is disposed parallel to the display module 100, so that the sensor 300 fixed on the fixing unit 232 directly faces the display module 100 to collect the light signal from the display module 100. Meanwhile, the fixing unit 232 is disposed parallel to the display module 100, so that the appearance of the display module 100 is exquisite and consistent. Generally, a portion of the housing 220 facing the user is also disposed parallel to the display module 100, so as to further ensure the aesthetic appearance of the medical display.

In this embodiment, the display module 100 includes a plurality of pixels, each of which includes sub-pixel units for displaying different colors; and a detection range of the sensor 300 in a light emitting layer covers all sub-pixel units of at least one pixel.

The sensor 300 faces the whole pixel to be collected of the display module 100, and if only the content of a sub-pixel unit is collected, the detection effect is affected. The detection range of sensor 300 in the light emitting layer covers all sub-pixel units of at least one pixel, so that the content from at least one whole pixel can be collected for analysis when receiving content of the pixel, and thus the measurement result can be obtained more accurately.

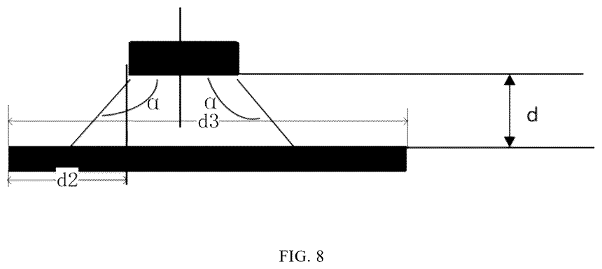

In the present embodiment, referring to FIG. 8, an angle between an edge of the sensor 300 and an edge of the auxiliary light emitting region 120 close to the edge of the sensor 300 is greater than or equal to 100° and less than or equal to 180°.

It is found through a large number of experiments that, when the angle α between the edge of the sensor 300 and the edge of the auxiliary light emitting region 120 is less than or equal to 100°, the number of the pixels of the display module 100 covered by the sensor 300 is not enough for detection, and when the angle α between the edge of the sensor 300 and the edge of the auxiliary light emitting region 120 is greater than or equal to 180°, the display module 100 is infinitely long. Preferably, the angle α is selected to be 150° in the present disclosure, so that the enough number of the pixels can be covered by the sensor 300 for detection, and the length of the display module 100 can further be ensured. In other embodiments, alternatively, the angle α may be selected to be 120° or 140°.

Referring to FIG. 8, a minimum distance from the sensor 300 to the display module 100 is greater than or equal to 0.2 mm and less than or equal to 5 mm.

It is further found through a large number of experiments that, when the minimum distance d from the sensor 300 to the display module 100 is less than or equal to 0.2 mm, the number of pixels, the contents of which can be received by the sensor 300, is not enough for detection, and when the minimum distance d from the sensor 300 to the display module 100 is greater than or equal to 5 mm, the frame 200 for fixing the sensor 300 is too thick, which results in poor appearance. Preferably, the minimum distance d from the sensor 300 to the display module 100 is greater than or equal to 0.5 mm and less than or equal to 2 mm, and with such range, the sensor 300 can detect enough pixels and the thickness of the frame 200 can further be ensured.

Referring to FIG. 8, a minimum distance from an edge of an orthographic projection of the sensor 300 on the display module 100 to the edge of the auxiliary light emitting region 120 is greater than or equal to 0.2 mm and less than or equal to 8 mm. Further, it is found that, when the minimum distance d2 from the edge of the orthographic projection of the sensor 300 on the display module 100 to the edge of the auxiliary light emitting region 120 is less than or equal to 0.2 mm, the number of pixels, the contents of which can be received by the sensor 300, is not enough for detection, and when the minimum distance d2 from the edge of the orthographic projection of the sensor 300 on the display module 100 to the edge of the auxiliary light emitting region 120 is greater than or equal to 8 mm, the auxiliary light emitting region 120 may be too long. Preferably, when the minimum distance d2 from the edge of the orthographic projection of the sensor 300 on the display module 100 to the edge of the auxiliary light emitting region 120 is greater than or equal to 0.866 mm and less than or equal to 3.4 mm, on one hand, the sensor 300 can detect enough number of the pixels, and the auxiliary light emitting region 120 can further be ensured not to be too long.

Referring to FIG. 8, a length from an end of the auxiliary light emitting region 120 away from the normal light emitting region 110 to an edge of the normal light emitting region 110 is greater than or equal to 1 mm and less than or equal to 15 mm.

When the length d3 from the end of the auxiliary light emitting region 120 away from the normal light emitting region 110 to the edge of the normal light emitting region 110 is less than or equal to 1 mm, the sensor 300 cannot detect enough number of the pixels, and when the length d3 from the end of the auxiliary light emitting region 120 away from the normal light emitting region 110 to the edge of the normal light emitting region 110 is greater than or equal to 15 mm, the auxiliary light emitting region 120 is too long. It is found through a lot of experiments that, when the length d3 from the end of the auxiliary light emitting region 120 away from the normal light emitting region 110 to the edge of the normal light emitting region 110 is greater than or equal to 4 mm and less than or equal to 7.5 mm, on one hand, the sensor 300 can detect enough number of the pixels, and the auxiliary light emitting region 120 can be further ensured not to be too long.

In addition, in the main design scheme for the sensor 300, the sensor 300 is placed in a space at the edge of the whole machine, for example, the sensor may be placed at any position of any one of an upper frame, a lower frame, a left frame and a right frame of the whole machine, preferably in a region located in the middle of the frame and occupying ⅔ of the length of the frame. This is because the closer to the corner of the whole machine the sensor is, the worse the brightness stability and uniformity are.

In the present disclosure, the structure embodiments and the method embodiments may complement each other without conflict.

The terms “first” and “second” are used for descriptive purposes only and are not to be construed as indicating or implying relative importance or to implicitly indicate the number of technical features indicated. Thus, a feature defined with “first” or “second” may explicitly or implicitly include one or more of the features. In the description of the present disclosure, the meaning of “a plurality” or “several” is two or more unless otherwise specified.

Other embodiments of the present disclosure will be apparent to one of ordinary skill in the art from consideration of the specification and practice of the content disclosed herein. The present disclosure is intended to cover any variations, uses, or adaptations of the present disclosure, and these variations, uses, or adaptations follow the general principle of the present disclosure and include the common general knowledge or the general technical means in the art, which is not disclosed in the present disclosure. The specification and embodiments are considered as exemplary only, with a true scope and spirit of the present disclosure being indicated by the claims.

It will be understood that the present disclosure is not limited to the precise arrangements that have been described above and shown in the drawings, and that various modifications and changes may be made without departing from the scope of the present disclosure. The scope of the present disclosure is limited only by the appended claims.

1. A medical display apparatus, comprising:
a display module comprising a normal light emitting region and an auxiliary light emitting region on a side of the normal light emitting region;
a frame configured to fix the display module and comprising a fixing region, wherein an orthographic projection of the fixing region along a thickness direction at least partially covers the auxiliary light emitting region; and
a sensor in the fixing region of the frame and directly facing the auxiliary light emitting region, and configured to receive a light signal emitted from the auxiliary light emitting region.

2. The medical display apparatus according to claim 1, wherein the display module comprises a backlight source module extending from the normal light emitting region to the auxiliary light emitting region, and a portion of the backlight source module in the light emitting region and another portion of the backlight source module in the auxiliary light emitting region are configured to be turned on or off synchronously.

3. The medical display apparatus according to claim 2, wherein the display module further comprises a display panel on the backlight source module and extending from the normal light emitting region to the auxiliary light emitting region, and the sensor faces a portion of the display panel in the auxiliary light emitting region to receive a light signal emitted from a whole structure formed by the backlight source module and the display panel.

4. The medical display apparatus according to claim 3, wherein the display panel comprises a first display unit and a second display unit, the first display unit is in the normal light emitting region, the second display unit is in the auxiliary light emitting region, and the first display unit and the second display unit operate synchronously or independently.

5. The medical display apparatus according to claim 4, wherein the medical display apparatus has a normal display state and a detection state;
in a case where the medical display apparatus is in the normal display state, the first display unit is in a light-transmitting state, light emitted from the backlight source module is emitted outwards by transmitting through the first display unit, and the second display unit is in a light-shielding state, and light emitted from the backlight source module is not transmitted through the second display unit; and
in a case where the medical display is in the detection state, the second display unit is in a light-transmitting state, and light emitted from the backlight source module is emitted outwards by transmitting through the second display unit and received by the sensor.

6. The medical display apparatus according to claim 4, wherein the frame comprises a light-shielding plate on a side of the display panel away from the backlight source module, and a minimum distance from an edge of the light-shielding plate to the first display unit is greater than or equal to 0.1 mm and/or less than or equal to 5 mm.

7. The medical display apparatus according to claim 2, wherein the display module further comprises a display panel on the backlight source module and in the normal light emitting region, an orthographic projection of the display panel on the backlight source module does not overlap with an orthographic projection of the auxiliary light emitting region on the backlight source module, and the sensor faces a portion of the backlight plate in the auxiliary light emitting region to receive a light signal emitted from the backlight source module.

8. The medical display apparatus according to claim 1, wherein the frame comprises a housing, the housing comprises a fixing plate located outside the display module, an orthographic projection of the fixing plate in the thickness direction is at least partially located in the auxiliary light emitting region, a portion of the fixing plate in the auxiliary light emitting region serves as the fixing region, and the sensor is on the fixing plate.

9. The medical display apparatus according to claim 1, wherein the frame comprises a housing and an auxiliary housing, the auxiliary housing comprises an extension unit and a fixing unit connected to each other, the extension unit is in the housing, and the fixing unit is exposed outside the housing; and
an orthographic projection of the fixing unit in the thickness direction is at least partially located in the auxiliary light emitting region, the fixing unit serves as the fixing region, and the sensor is on the fixing unit.

10. The medical display apparatus according to claim 8, wherein
the sensor is on the fixing plate, and the fixing plate and the display module are parallel to each other; or
the sensor is on the fixing unit, and the fixing unit and the display module are parallel to each other.

11. The medical display apparatus according to claim 1, wherein the display module comprises a plurality of pixels, each pixel comprising sub-pixel units for displaying different colors; and
a detection range of the sensor in a light emitting layer covers all sub-pixel units in at least one pixel.

12. The medical display apparatus according to claim 1, wherein an angle between an edge of the sensor and an edge of the auxiliary light emitting region is greater than or equal to 100° and less than or equal to 180°; and/or
a minimum distance from the sensor to the display module is greater than or equal to 0.2 mm and less than or equal to 5 mm; and/or
a minimum distance from an edge of an orthographic projection of the sensor on the display module to the edge of the auxiliary light emitting region is greater than or equal to 0.2 mm and less than or equal to 8 mm; and/or
a length from an end of the auxiliary light emitting region away from the normal light emitting region to an edge of the normal light emitting region is greater than or equal to 1 mm and less than or equal to 15 mm.

13. The medical display apparatus according to claim 9, wherein
the sensor is on the fixing plate, and the fixing plate and the display module are parallel to each other; or
the sensor is on the fixing unit, and the fixing unit and the display module are parallel to each other.
